(19) United States
(12) Patent Application Publication
(10) Pub. No.: US 2023/0149086 A1
(43) Pub. Date: May 18, 2023

(54) MAXILLARY SINUS LIFTING SIMULATION METHOD AND APPARATUS THEREFOR

(71) Applicant: OSSTEM IMPLANT CO., LTD., Seoul (KR)

(72) Inventors: Won Hyeong JOO, Seoul (KR); Do Hyun LEE, Seoul (KR); Kyoo Ok CHOI, Seoul (KR)

(73) Assignee: OSSTEM IMPLANT CO., LTD., Seoul (KR)

(21) Appl. No.: 17/917,428

(22) PCT Filed: Mar. 18, 2021
(86) PCT No.: PCT/KR2021/003319

§ 371 Date: Oct. 6, 2022

(30) Foreign Application Priority Data
May 19, 2020 (KR) 10-2020-0059741

(51) Int. Cl. A61B 34/10

(52) U.S. Cl. CPC A61B 34/10; A61B 2034/104; A61B 2034/105; A61C 8/0092

(57) ABSTRACT

A maxillary sinus lifting simulation method and an apparatus therefor are disclosed. According to the maxillary sinus lifting simulation method and apparatus therefor according to one embodiment, in a case in which an implant placed while performing implant placement simulation in a dental image involves the maxillary sinus, a maxillary sinus lifting simulation is automatically performed to display maxillary sinus lifting simulation information in the dental image. In this way, convenience of use can be improved, and errors can be reduced.

### TECHNICAL FIELD

The present disclosure relates to a dental image processing technology, and more particularly, to a technology of simulating maxillary sinus lifting in a dental image.

### BACKGROUND ART

An implant is a dental prosthesis used to fix an artificial tooth by placing a implant into or on the jawbone to support a crown for filling a damaged tooth. For successful implant surgery, it is important to firmly fix an implant to a bone. However, when the bone tissue is weak or the bone thickness is insufficient at a position where the implant will be placed, implant surgery is performed after reinforcing the bone tissue by the grafting of an autogenous bone, an artificial bone, a synthetic bone, or the like.

In particular, when performing implant surgery at a position below the maxillary sinus, in many cases, the bone thickness is insufficient relative to an implant length, which makes it difficult to place the implant. Here, when implant surgery is performed despite the insufficient bone thickness, since the surrounding bone tissue cannot sufficiently support the placed implant, a problem that the implant falls out, or the surrounding bone tissue breaks down during masticatory movement, may occur. In order to prevent such a problem, when the thickness of maxillary bone is insufficient, maxillary sinus lifting in which the maxillary sinus membrane is elevated and then a bone is grafted into the maxillary sinus to secure an implant placement space is performed. The maxillary sinus membrane tissue is a tissue formed in the maxillary sinus to control humidity in the nasal cavity and control resonance during vocalization.

However, in general dental image processing software, during implant placement simulation, a user should directly check whether maxillary sinus involvement by an implant occurs. In this case, it is inconvenient to use, and during maxillary sinus lifting surgery, there is a risk of errors due to a user performing the surgery based on the user's own experience and judgement.

### DISCLOSURE

### Technical Problem

One embodiment of the present disclosure proposes a maxillary sinus lifting simulation method and an apparatus therefor capable of, during i placement simulation, automatically performing maxillary sinus lifting simulation to inform a user of whether maxillary sinus involvement by an implant occurs, thereby improving convenience of use and reducing errors.

### Technical Solution

A maxillary sinus lifting simulation method according to one embodiment includes an operation of performing implant placement simulation in a dental image of a patient, an operation of, during the implant placement simulation, determining whether maxillary sinus lifting is necessary due to maxillary sinus involvement by an implant, an operation of automatically performing maxillary sinus lifting simulation only in a case in which the maxillary sinus lifting is necessary and automatically displaying maxillary sinus lifting simulation information, which includes information on an area where maxillary sinus involvement occurs based on the implant and information on an amount of bone graft, in the dental image, and an operation of, in response to a change in an implant placement situation, changing the maxillary sinus lifting simulation information according to the changed implant placement situation and automatically displaying the changed maxillary sinus lifting simulation information in the dental image.

The operation of the determining of whether the maxillary sinus lifting is necessary may include an operation of setting a maxillary sinus simulation area based on the implant placed in the dental image, an operation of setting an implant involvement area within the set maxillary sinus simulation area, an operation of determining whether the implant involves the implant involvement area in an implant placement process, and an operation of, in response to the implant involving the implant involvement area, determining that the maxillary sinus lifting is necessary.

The operation of the setting of the maxillary sinus simulation area may include an operation of setting the maxillary sinus simulation area in a semispherical shape having, as a radius, a length from a reference point of the implant as a start point to a point at a predetermined distance from an upper end of the implant.

In the operation of the setting of the maxillary sinus simulation area, in a case in which two implants are consecutively placed, in response to two maxillary sinus simulation areas at least partially overlapping with each other, end points of the two maxillary sinus simulation areas may be connected to each other to set a single maxillary sinus simulation area, and in the operation of the setting of the implant involvement area, a single implant involvement area that the two implants involve may be set within the set single maxillary sinus simulation area.

The operation of the setting of the implant involvement area may include an operation of calculating a density value of the set maxillary sinus simulation area and an operation of setting an area having a density value less than a predetermined density value in the maxillary sinus simulation area as the implant involvement area.

The operation of the automatically displaying of the maxillary sinus lifting simulation information in the dental image may include an operation of displaying the implant involvement area in the dental image and an operation of calculating and displaying a volume of the implant involvement area.

In the operation of the changing of the maxillary sinus lifting simulation information and the automatically displaying of the changed maxillary sinus lifting simulation information in the dental image, in response to a change in implant information, the information on the area where the maxillary sinus involvement occurs based on the implant and the information on the amount of bone graft may be changed to correspond to the changed implant information and may be automatically displayed in the dental image.

In the operation of the changing of the maxillary sinus lifting simulation information and the automatically displaying of the changed maxillary sinus lifting simulation information in the dental image, in response to movement of the implant, the maxillary sinus simulation area may be moved together, in response to a change in a length of the implant, the radius of the maxillary sinus simulation area may be changed, and then the information on the area where the maxillary sinus involvement occurs based on the implant and the information on the amount of bone graft may be changed to correspond to the movement of the maxillary sinus simulation area or the change in the radius and may be automatically displayed in the dental image.

The maxillary sinus lifting simulation method may further include an operation of providing a maxillary sinus lifting simulation setting screen for setting at least one of an interface for setting whether to use a maxillary sinus lifting simulation, an interface for setting a display color of an implant involvement area, an interface for setting a length from an upper end of the implant to a maxillary sinus simulation area, an interface for setting opacity of the implant involvement area, and an interface for setting a reference point of the implant.

The maxillary sinus lifting simulation method may further include an operation of receiving a manipulation signal for user setting relating to area showing and area hiding and an operation of, in a case in which an area showing setting is selected according to the user setting, displaying the implant involvement area and information on a predicated amount of bone graft in the dental image and, in a case in which an area hiding setting is selected, not displaying the implant involvement area and the information on the predicated amount of bone graft in the dental image.

A maxillary sinus lifting simulation apparatus according to one embodiment includes a data acquisition device configured to acquire a dental image of a patient, a controller configured to perform implant placement simulation in the dental image of the patient, during the implant placement simulation, determine whether maxillary sinus lifting is necessary due to maxillary sinus involvement by an implant, automatically perform maxillary sinus lifting simulation only in a case in which the maxillary sinus lifting is necessary, provide maxillary sinus lifting simulation information, which includes information on an area where maxillary sinus involvement occurs based on the implant and information on an amount of bone graft, and, in response to a change in an implant placement situation, change the maxillary sinus lifting simulation information according to the changed implant placement situation, and an output device configured to automatically display the maxillary sinus lifting simulation information in the dental image.

The controller may set a maxillary sinus simulation area based on the implant placed in the dental image, set an implant involvement area within the set maxillary sinus simulation area, and then determine whether the implant involves the implant involvement area in an implant placement process, and, in response to the implant involving the implant involvement area, determine that the maxillary sinus lifting is necessary.

The controller may set the maxillary sinus simulation area in a semispherical shape having, as a radius, a length from a reference point of the implant as a start point to a point at a predetermined distance from an upper end of the implant, and, in a case in which two implants are consecutively placed, in response to two maxillary sinus simulation areas at least partially overlapping with each other, the controller may connect end points of the two maxillary sinus simulation areas to each other to set a single maxillary sinus simulation area and may set a single implant involvement area that the two implants involve within the set single maxillary sinus simulation area.

The output device may display the implant involvement area and a volume thereof in the dental image and, in response to a change in implant information, change the implant involvement area and the volume thereof to correspond to the changed implant information and display the changed implant involvement area and volume thereof in the dental image.

The output device may provide a maxillary sinus lifting simulation setting screen for setting at least one of an interface for setting whether to use maxillary sinus lifting simulation, an interface for setting a display color of the implant involvement area, an interface for setting a length from the upper end of the implant to the maxillary sinus simulation area, an interface for setting opacity of the implant involvement area, and an interface for setting the reference point of the implant.

### Advantageous Effects

According to a maxillary sinus lifting simulation method and apparatus therefor according to one embodiment, during implant placement simulation, maxillary sinus lifting simulation is automatically performed to provide information on an area where maxillary sinus lifting is necessary and information on an amount of bone graft to a user. In this way, the user can establish a plan for maxillary sinus lifting and bone grafting surgery by referring to the provided pieces of information.

Since the information on the area where maxillary sinus lifting is necessary and the information on the amount of bone graft are proposed to the user, the user can prepare for surgery after checking a necessary amount of bone graft in advance in the process of establishing the surgical plan and can make good use of the pieces of information even during the actual surgery.

Also, since maxillary sinus lifting simulation information is automatically changed and provided in response to a change in an implant placement situation such as the position or length of an implant, it helps the user reduce errors while easily using the provided maxillary sinus lifting simulation information.

Further, since a screen for setting maxillary sinus lifting simulation is provided and a user interface is provided on the screen, user convenience can be improved.

### DESCRIPTION OF DRAWINGS

FIG. 1 is a view illustrating a configuration of a maxillary sinus lifting simulation apparatus according to one embodiment of the present disclosure.

FIG. 2 is a view illustrating image screens according to a maxillary sinus lifting simulation process according to one embodiment of the present disclosure.

FIG. 3 is a view illustrating image screens according to a maxillary sinus lifting simulation process according to another embodiment of the present disclosure.

FIG. 4 is a view illustrating a setting screen for maxillary sinus lifting simulation and a use example thereof according to one embodiment of the present disclosure.

FIG. 5 is a view illustrating image screens showing movement of a maxillary sinus simulation area according to movement of an implant according to one embodiment of the present disclosure.

FIG. 6 is a view illustrating image screens displaying movement of an implant involvement area and a change in an amount of bone graft according to movement of an implant.

FIG. 7 is a view illustrating image screens of the implant involvement area according to a change in the length of the implant according to one embodiment of the present disclosure.

FIG. 8 is a view illustrating a screen automatically displaying maxillary sinus lifting simulation information in a 3D image according to one embodiment of the present disclosure.

FIG. 9 is a view illustrating image screens in a case in which screen showing is selected by a user according to one embodiment of the present disclosure.

FIG. 10 is a view illustrating image screens in a case in which screen hiding is selected by the user according to one embodiment of the present disclosure.

FIG. 11 is a view illustrating image screens for maxillary sinus lifting simulation according to one embodiment of the present disclosure.

FIG. 12 is a view illustrating a flowchart of a maxillary sinus lifting simulation method according to one embodiment of the present disclosure.

### MODES OF THE INVENTION

Advantages and features of the present disclosure and methods of achieving the same should become clear from embodiments described in detail below with reference to the accompanying drawings. However, the present disclosure is not limited to the embodiments disclosed below and may be implemented in various different forms. The embodiments herein only make the disclosure of the present disclosure complete and are provided to completely inform those of ordinary skill in the art to which the present disclosure pertains of the scope of the disclosure. The present disclosure is defined only by the scope of the claims. Like components are denoted by like reference numerals throughout the specification.

In describing the embodiments of the present disclosure, when detailed description of a known function or component is determined as having the possibility of unnecessarily obscuring the gist of the present disclosure, the detailed description will be omitted, and the terms used herein are terms defined in consideration of functions in the embodiments of the present disclosure and may vary according to an intention or practice of a user or an operator. Therefore, the terms should be defined on the basis of the content throughout the specification.

Since combinations of blocks of the accompanying block diagram and operations of the accompanying flowchart may be performed by computer program instructions (execution engines) and the computer program instructions may be embedded in processors of general purpose computers, special purpose computers, or other programmable data processing apparatuses, the instructions that are executed by the processors of the computers or other programmable data processing apparatuses generate means for performing functions described in the blocks of the block diagram or the operations of the flowchart.

Since the computer program instructions may be stored in computer usable or computer readable memories capable of being used in computers or other programmable data processing apparatuses to realize functions in particular manners, the instructions stored in the computer usable or computer readable memories can also produce manufacturing items that include instruction means configured to perform the functions described in the blocks of the block diagram or the operations of the flowchart.

In addition, since the computer program instructions may be embedded in the computers or other programmable data processing apparatuses, the instructions, which perform a series of operations in the computers or the other programmable data processing apparatuses to generate computer-executed processes and execute the computers or the other programmable data processing apparatuses, may also provide operations for executing the functions described in the blocks of the block diagram and the operations of the flowchart.

In addition, the blocks or operations may represent portions of modules, segments, or codes including one or more executable instructions for executing specified logical functions, and in some alternative embodiments, the functions described in the blocks or operations may also be performed out of order. For example, two blocks or operations which are sequentially described may also be simultaneously performed, or the blocks or operations may also be performed in reverse order of the corresponding functions as necessary.

Hereinafter, embodiments of the present disclosure will be described in detail with reference to the accompanying drawings. However, the embodiments of the present disclosure which will be described below may be modified in several different forms, and the scope of the present disclosure is not limited to the embodiments to be described below. The embodiments of the present disclosure are provided to fully explain the present disclosure to those of ordinary skill in the art.

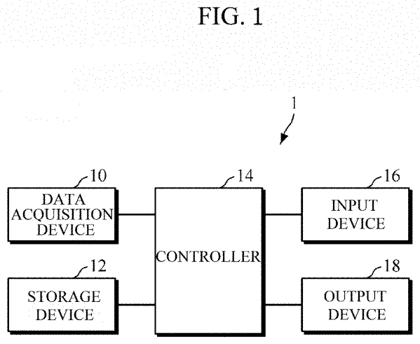

FIG. 1 is a view illustrating a configuration of a maxillary sinus lifting simulation apparatus (hereinafter referred to as a “simulation apparatus”) according to one embodiment of the present disclosure.

Referring to FIG. 1, a simulation apparatus 1 according to one embodiment establishes an implant placement plan by designing parts such as a crown, an implant, and an abutment for dental treatment. A user may simulate the established implant placement plan and then perform implant surgery. In particular, among surgical cases, when planning implant placement in a maxillary tooth, implant surgery may be performed by checking a maxillary sinus area in a dental image to prevent perforation of the maxillary sinus, predicting a case in which maxillary sinus lifting is necessary, performing the maxillary sinus lifting when necessary, and then placing the implant.

Segmentation and volume measurement functions are provided for a maxillary sinus area through a dental image program using a 3D volume rendering technique of computed tomography (CT) image data, which is a dental image. In this way, the user may check the maxillary sinus area in 2D/3D images. The simulation apparatus 1 according to one embodiment performs implant placement simulation for placing an implant in a dental image and, in a case in which the implant placed during the implant placement simulation involves the maxillary sinus, automatically performs maxillary sinus lifting simulation and displays maxillary sinus lifting simulation information in the dental image. The maxillary sinus lifting simulation information includes information on an area where maxillary sinus involvement occurs based on the implant and information on an amount of bone graft. The information on the amount of bone graft may be acquired by calculating the volume of an implant involvement area. In this way, by automatically displaying the implant involvement area when the implant involves the maxillary sinus, it is possible to address problems that may occur when the user performs maxillary sinus lifting and implant surgery based on the user's own experience and judgement.

The simulation apparatus 1 is an electronic apparatus that executes image processing software for performing simulation in a dental image. Examples of the electronic apparatus include a computer, a laptop, a tablet personal computer (PC), a smartphone, a mobile phone, a personal media player (PMP), a personal digital assistant (PDA), and the like. Examples of the image processing software include a guide design program, implant simulation software, scan software, computer-aided design (CAD) software, and the like. Also, the image processing software may be applied as software for processing general dental images other than those for use in dental implant surgery.

A dental guide design process using the image processing software consists of registering a patient to receive surgery, acquiring CT data and oral scan data of the registered patient, matching the CT data and the oral scan data, generating a dental arch line in the matched image data, generating a panoramic image using the dental arch line, setting the position and size of a crown model in the oral scan data of the patient, setting the position of an implant structure including an implant in the CT data of the patient, designing the shape of a guide, and outputting the final guide. The present disclosure relates to a technology of automatically providing maxillary sinus lifting simulation information in response to maxillary sinus involvement by an implant in an implant placement simulation process during the above process.

Referring to FIG. 1, the simulation apparatus 1 according to one embodiment includes a data acquisition device 10, a storage device 12, a controller 14, an input device 16, and an output device 18.

The data acquisition device 10 acquires dental image data from teeth including a damaged target tooth of a patient. Examples of the dental image data include CT image data and oral scan data of the patient.

The oral scan data is data containing information on the actual teeth including the damaged tooth and may be 3D information. The oral scan data may be acquired by scanning a plaster model of an oral cavity of the patient with a 3D scanner. As another example, the oral scan data may be acquired by scanning the inside of the oral cavity of the patient using a 3D intra-oral scanner. The acquired oral scan data may be stored in the storage device 12.

The CT image data may be acquired by generating tomographic images of the patient's head using CT, segmenting boundaries of teeth in each tomographic image, and then combining the segmented boundaries into one. The oral scan data and the CT image data include an image obtained by imaging maxillary teeth from below the maxillary teeth with the patient's mouth open, an image obtained by imaging mandibular teeth above the mandibular teeth with the patient's mouth open, an image obtained by imaging a local area with the patient's mouth closed, an oral radiograph, and the like. The obtained CT image data may be stored in the storage device 12.

Various pieces of data such as information necessary to perform operations of the simulation apparatus 1 and information generated according to performing the operations are stored in the storage device 12. Pieces of oral scan data and CT image data of individual patients may be stored in the storage device 12 according to one embodiment, and during dental treatment simulation, oral scan data and CT image data of a specific patient, among all the pieces of oral scan data and CT image data, may be provided to the controller 14 according to a user request. Here, maxillary teeth arrangement images and mandibular teeth arrangement images of individual patients may be stored in the storage device 12, and a maxillary teeth arrangement image and a mandibular teeth arrangement image which match the oral scan data and CT image data of the specific patient may be provided to the controller 14 according to a user request.

The controller 14 controls each functional component while performing implant placement simulation and maxillary sinus lifting simulation through control using a computer program. The controller 14 manages screen information displayed on a screen through the output device 18 and performs the implant placement simulation in which a virtual implant object is placed in a dental image. The dental image in which the virtual implant object is placed refers to a multi-dimensional image such as a 2D image or a 3D image which shows teeth arrangement of a patient and is generated to establish an implant surgery plan. Various types of images such as an x-ray image, a CT image, a magnetic resonance imaging (MRI) image, a panoramic image, an oral scan image, an image generated through reconstruction, and an image obtained by matching a plurality of images may be utilized in the implant surgery plan.

The controller 14 according to one embodiment automatically performs maxillary sinus lifting simulation during the implant placement simulation. For example, when the maxillary sinus lifting is necessary due to maxillary sinus involvement by an implant, the controller 14 performs the maxillary sinus lifting simulation and automatically displays maxillary sinus lifting simulation information in a dental image. The maxillary sinus lifting simulation information includes information on an area where maxillary sinus involvement occurs based on the implant and information on an amount of bone graft. The information on the amount of bone graft may be expressed as a volume value of an implant involvement area.

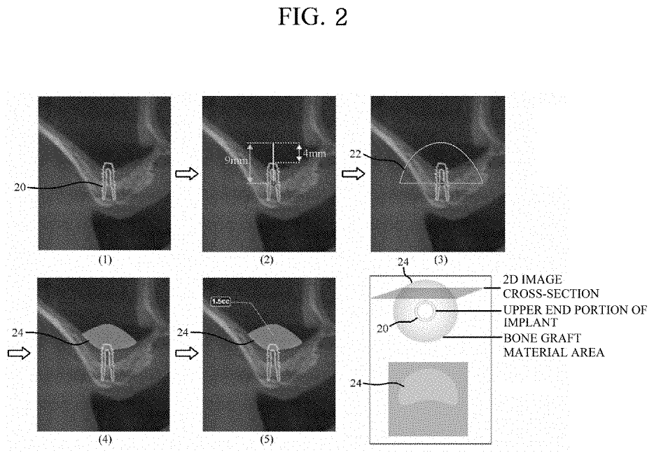

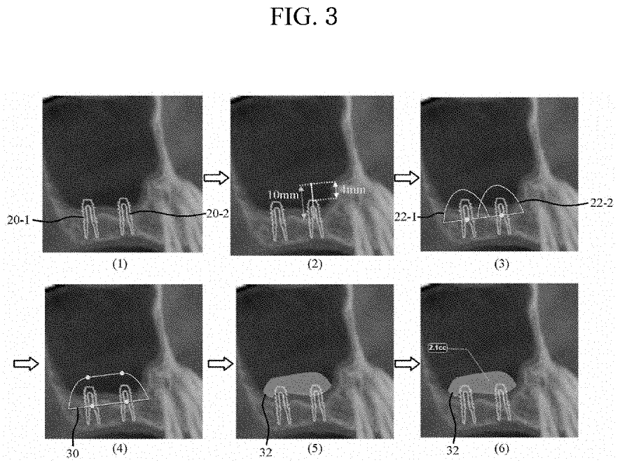

In order to determine whether the maxillary sinus lifting is necessary, the controller 14 sets each of a maxillary sinus simulation area and the implant involvement area. The maxillary sinus simulation area refers to a semispherical area having, as a radius, a length from a reference point of the implant as a start point to a point at a predetermined distance from an upper end of the implant. The implant involvement area refers to an area having a density value less than a predetermined density value in the maxillary sinus simulation area. In response to the implant involving the implant involvement area, the controller 14 may determine that the maxillary sinus lifting is necessary. The setting of the maxillary sinus simulation area and the implant involvement area will be described below with reference to FIGS. 2 and 3.

In response to a change in an implant placement situation, the controller 14 may change the maxillary sinus lifting simulation information according to the changed implant placement situation and automatically display the changed maxillary sinus lifting simulation information in the dental image. For example, in response to a change in implant information, the controller 14 changes the information on the area where the maxillary sinus involvement occurs based on the implant and the information on the amount of bone graft to correspond to the changed implant information and automatically displays the changed information on the area where the maxillary sinus involvement occurs based on the implant and information on the amount of bone graft in the dental image. The implant information may include a length, a diameter, a slope, and the like of the implant. In response to movement of the implant, the controller 14 may move the maxillary sinus simulation area together, and in response to a change in the length of the implant, the controller 14 may change the radius of the maxillary sinus simulation area. According to a change in the maxillary sinus simulation area, the controller 14 may also change the implant involvement area and the information on the amount of bone graft and display the changed implant involvement area and information on the amount of bone graft on the screen.

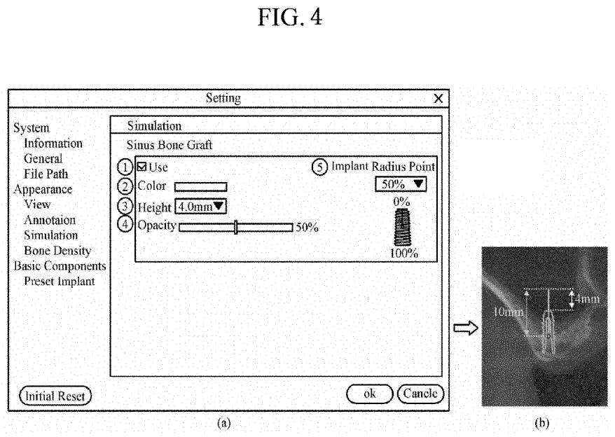

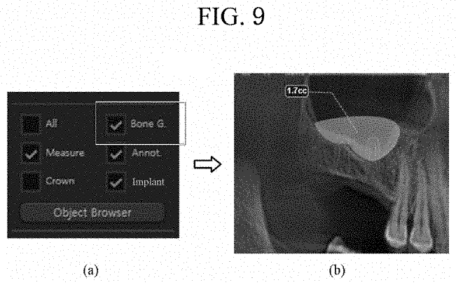

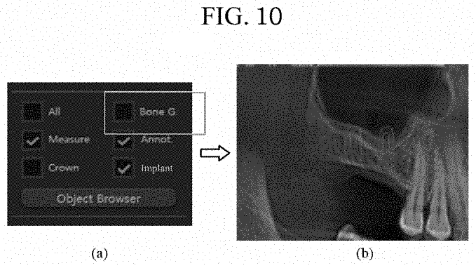

The input device 16 receives a manipulation signal from a user. For example, the input device 16 receives a manipulation signal for user setting on a maxillary sinus lifting simulation setting screen. The maxillary sinus lifting simulation setting screen includes an interface for setting whether to use maxillary sinus lifting simulation, an interface for setting a display color of the implant involvement area, an interface for setting a length from the upper end of the implant to the maxillary sinus simulation area, an interface for setting opacity of the implant involvement area, an interface for setting the reference point of the implant, and the like. The maxillary sinus lifting simulation setting screen will be described below with reference to FIG. 4. The input device 16 may receive a manipulation signal for user setting relating to area showing and area hiding. The setting of the area showing and area hiding will be described below with reference to FIGS. 9 and 10.

The output device 18 displays a screen. Here, the output device 18 may display a dental image for placing a virtual implant object, an implant placement simulation screen on which the implant is placed in the dental image, a maxillary sinus lifting simulation screen, and the like.

The output device 18 may display the implant involvement area to be visually identifiable through the maxillary sinus lifting simulation screen and may also display information on the volume of the implant involvement area. Here, in response to a change in the implant information, the output device 18 may change the implant involvement area and the volume thereof to correspond to the changed implant information and display the changed implant involvement area and volume thereof.

FIG. 2 is a view illustrating image screens according to a maxillary sinus lifting simulation process according to one embodiment of the present disclosure.

Referring to FIG. 2, in (1), software places an implant 20 in a dental image of a patient. A CT image may be used to set an implant placement position. The CT image includes a 3D image, a 2D image, and a panoramic image. The 2D image includes an axial image, a sagittal image or a cross image, a coronal image, and the like. In an implant placement operation, the implant may be automatically placed using a reference axis of a crown, adjacent teeth, and bone density information. Using setting information for each tooth number stored in an implant library, the implant 20 of a set size, that is, a set length and a diameter, may be placed at a position corresponding to a selected tooth number.

Then, in (2), the software calculates a length (e.g., 9 mm) from a reference point of the placed implant 20 as a start point to a point at a predetermined distance (e.g., 4 mm) from an upper end of the implant 20 and sets the calculated length as a radius. For example, as illustrated in FIG. 2, in a case in which the length of the implant 20 is 10 mm and the predetermined distance is 4 mm, the length 9 mm, which is obtained by adding the value of the predetermined distance 4 mm to 5 mm which is a value of a length from a central point of the implant 20 to the upper end of the implant 20, is set as the radius. Although the central point is set as the reference point in FIG. 2, the reference point may be changed by user setting. The predetermined distance from the upper end of the implant 20 may also be changed by user setting.

Then, in (3), the software sets a maxillary sinus simulation area 22 in a semispherical shape having the calculated length (e.g., 9 mm) as the radius. The maxillary sinus simulation area 22 is generated in a semicircular shape in a 2D cross-section image and is generated in the semispherical shape in a 3D image.

Then, in (4), the software sets an area having a density value less than a predetermined density value in the set maxillary sinus simulation area 22 as an implant involvement area 24 and then, in a case in which the implant 20 involves the implant involvement area 24, displays the implant involvement area 24 on the screen. Here, the implant involvement area 24 is provided in the form of identifiable visual information on the screen to allow the user to check the implant involvement area 24. The identifiable visual information may be displayed using various methods, e.g., displayed with a different color, distinguished by a pattern, or indicated with a separate mark, an outline, or a leader line. The Hounsfield Unit (HU) value may be used for the density value. Here, the implant involvement area 24 may be displayed to have a predetermined opacity value.

Then, in (5), the software calculates and displays the volume of the implant involvement area 24. The volume of the implant involvement area 24 may be provided as a numerical value. The unit of the volume of the implant involvement area 24 may be cc or mm³ but is not limited thereto. In FIG. 2, “1.5 cc” is displayed on the screen. The volume information may be displayed in a 2D image, a 3D image, or on a separate screen. Using the volume information of the implant involvement area 24, the user may recognize the amount of bone graft material necessary for the corresponding area. The bone graft material is a material developed to, in the case of bone loss due to disease or trauma, fill the bone in an area where the bone is missing. When, through surgery, the bone graft material is placed into an area where bone is missing, new bone grows around the bone graft material. In dentistry, the bone graft material is used as a material for filling an area where bone is missing when placing an implant.

FIG. 3 is a view illustrating image screens according to a maxillary sinus lifting simulation process according to another embodiment of the present disclosure.

While the case described above with reference to FIG. 2 is a case in which a single implant is placed, FIG. 3 corresponds to a case in which two or more implants are consecutively placed.

In (1), the software places implants in a dental image of a patient. Two or more implants, e.g., a first implant 20-1 and a second implant 20-2 as illustrated in FIG. 3, are consecutively placed.

In (2), when placing the two consecutive implants 20-1 and 20-2, the software calculates a length from a reference point of each of the implants 20-1 and 20-2 as a start point to a point at a predetermined distance from an upper end of each of the implants 20-1 and 20-2 and sets the calculated length as a radius. For example, in a case in which the length of the second implant 20-2 is 12 mm and the predetermined distance is 4 mm as illustrated in FIG. 3, the length 10 mm, which is obtained by adding the value of the predetermined distance 4 mm to 6 mm which is a value of a length from a central point of the second implant 20-2 to the upper end of the second implant 20-2, is set as the radius. Although the central point is set as the reference point in FIG. 3, the reference point may be changed by user setting. The predetermined distance from the upper end of each of the implants 20-1 and 20-2 may also be changed by user setting.

Then, in (3), the software sets maxillary sinus simulation areas 22-1 and 22-2 in semispherical shapes having the respective calculated lengths as the radius. The two maxillary sinus simulation areas 22-1 and 22-2 are generated in a semicircular shape in a 2D cross-section image and are generated in the semispherical shape in a 3D image. Here, the two maxillary sinus simulation areas 22-1 and 22-2 may at least partially overlap with each other.

In (4), in response to the two maxillary sinus simulation areas 22-1 and 22-2 at least partially overlapping with each other, the software connects end points of the two maxillary sinus simulation areas 22-1 and 22-2 to each other to generate a single maxillary sinus simulation area 30. For example, the software detects a first end point, where a vertical line segment starting from the central point of the first implant 20-1 meets a first maxillary sinus simulation area 22-1, and a second end point, where a vertical line segment starting from the central point of the second implant 20-2 meets a second maxillary sinus simulation area 22-2, and connects the first end point and the second end point. Here, the single maxillary sinus simulation area 30 which has a semicylindrical shape and a height equal to a height of a line segment generated by connecting the first end point and the second end point is newly generated.

Then, in (5), the software sets an area having a density value less than a predetermined density value in the set single maxillary sinus simulation area 30 as an implant involvement area 32 and then, in a case in which the implants 20-1 and 20-2 involve the implant involvement area 32, displays the implant involvement area 32 on the screen. Here, the implant involvement area 32 is provided in the form of identifiable visual information on the screen to allow the user to check the implant involvement area 32 from images. The identifiable visual information may be displayed using various methods, e.g., displayed with a different color, distinguished by a pattern, or indicated with a separate mark, an outline, or a leader line. The HU value may be used for the density value. Here, the implant involvement area 32 may be displayed to have a predetermined opacity value.

Then, in (6), the software calculates and displays the volume of the implant involvement area 32. The volume of the implant involvement area 32 may be provided as a numerical value. The unit of the volume of the implant involvement area 32 may be cc or mm³ but is not limited thereto. In FIG. 3, “2.1 cc” is displayed on the screen. The volume information may be displayed in a 2D image, a 3D image or on a separate screen. Using the volume information of the implant involvement area 32, the user may recognize the amount of bone graft necessary for the maxillary sinus lifting. Here, the volume information is information in which multiple pieces of information are combined into one.

FIG. 4 is a view illustrating a setting screen for maxillary sinus lifting simulation and a use example thereof according to one embodiment of the present disclosure.

In more detail, FIG. 4A illustrates a maxillary sinus lifting simulation setting screen, and FIG. 4B illustrates a use example of maxillary sinus lifting simulation setting.

The maxillary sinus lifting simulation setting screen includes: 1) an interface for setting whether to use maxillary sinus lifting simulation; 2) an interface for setting a display color of the implant involvement area; 3) an interface for setting a length from the upper end of the implant to the maxillary sinus simulation area; 4) an interface for setting opacity of the implant involvement area; and 5) an interface for setting the reference point of the implant. FIG. 4B illustrates an example in which the length set through the interface for setting the length from the upper end of the implant to the maxillary sinus simulation area (“3)”) is 4 mm, and a reference point set through the interface for setting the reference point of the implant (“5)”) is the central point of the implant that corresponds to 50%.

Through the maxillary sinus lifting simulation setting, the user can arbitrarily set whether to use maxillary sinus lifting simulation, the display color of the implant involvement area, the height from the end of the implant, the opacity of the expressed color, and a start point of the radius based on the implant. Through the setting, the user can deal with various parameters for each case and situation.

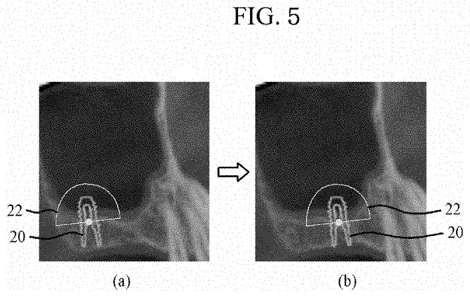

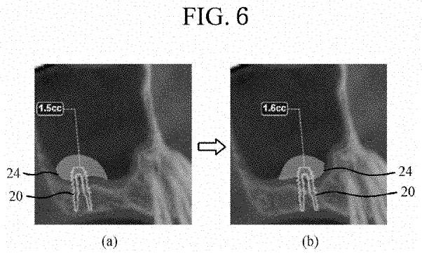

FIG. 5 is a view illustrating image screens showing movement of a maxillary sinus simulation area according to movement of an implant according to one embodiment of the present disclosure, and FIG. 6 is a view illustrating image screens displaying movement of an implant involvement area and a change in an amount of bone graft according to movement of an implant.

Referring to FIG. 5, as illustrated in FIG. 5A, the software generates the maxillary sinus simulation area 22 based on the implant 20. Then, as illustrated in FIG. 5B, in a case in which the implant 20 moves, the software also identically moves the maxillary sinus simulation area 22 according to the corresponding implant 20. The movement of the implant includes a change in the slope of the implant.

In response to the maxillary sinus simulation area 22 moving according to the movement of the implant 20 as illustrated in FIG. 5, the software also changes the position of the implant involvement area 24 and displays the changed position and also changes the volume value of the implant involvement area 24 and displays the changed volume value as illustrated in FIG. 6. For example, it can be seen from FIG. 6 that the volume value is changed from 1.5 cc in FIG. 6A to 1.6 cc in FIG. 6B.

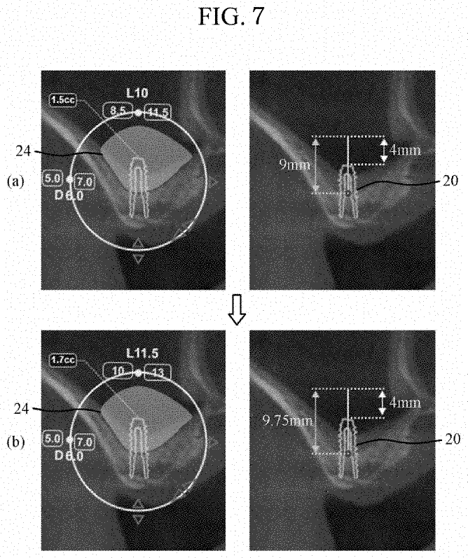

FIG. 7 is a view illustrating image screens of the implant involvement area according to a change in the length of the implant according to one embodiment of the present disclosure.

In more detail, FIG. 7A illustrates an image screen before a change in the length of the implant, and FIG. 7B illustrates an image screen after the change in the length of the implant.

Referring to FIG. 7A, in a case in which, based on the center line of the implant 20, a length from the central point of the implant 20 to a point at a predetermined distance (e.g., 4 mm) from the upper end of the implant 20 is designated as a length of a set area, before a change in the length of the implant, a maxillary sinus simulation area generated when an implant having a length of 10 mm is placed has a semispherical shape whose radius has a length of 9 mm. Therefore, a simulation is performed based on the semispherical shape whose radius is 9 mm, and the implant involvement area 24 and the volume value thereof (1.5 cc) are displayed based on the semispherical shape.

Referring to FIG. 7B, in a case in which the length of the implant is changed to 11.5 mm, the maxillary sinus simulation area has a semispherical shape whose radius has a length of 9.75 mm. Since the length of the radius of the maxillary sinus simulation area is increased by 0.75 mm as compared to when the length of the implant is 10 mm, the area of the displayed implant involvement area 24 is also larger, and the displayed volume thereof is also larger (is increased from 1.5 cc to 1.7 cc).

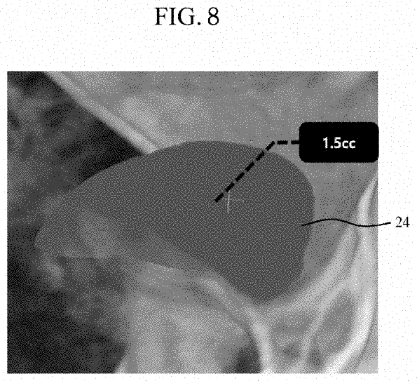

FIG. 8 is a view illustrating a screen automatically displaying maxillary sinus lifting simulation information in a 3D image according to one embodiment of the present disclosure.

Referring to FIG. 8, in a case in which, during the implant placement simulation, maxillary sinus involvement by an implant occurs, the software three-dimensionally displays maxillary sinus lifting simulation information, which includes the implant involvement area 24, on a 3D image screen. As in a 2D cross-section image, the implant involvement area 24 is provided in the form of identifiable visual information on the screen to allow the user to check the implant involvement area 24. The identifiable visual information may be displayed using various methods, e.g., displayed with a different color, distinguished by a pattern, or indicated with a separate mark, an outline, or a leader line. Here, the software may also display the volume value (e.g., 1.5 cc) of the implant involvement area 24.

FIG. 9 is a view illustrating image screens in a case in which screen showing is selected by a user according to one embodiment of the present disclosure.

Referring to FIG. 9, the user may set an implant involvement area to be shown on the screen by selecting area showing. For example, in a case in which the user has selected area showing on the setting screen as illustrated in FIG. 9A, the implant involvement area and information on a predicated amount of bone graft may be displayed together on an image screen of FIG. 9B.

FIG. 10 is a view illustrating image screens in a case in which screen hiding is selected by the user according to one embodiment of the present disclosure.

Referring to FIG. 10, the user may set an implant involvement area to be hidden on the screen by selecting area hiding. For example, in a case in which the user has selected area hiding on the setting screen as illustrated in FIG. 10A, the implant involvement area and information on a predicated amount of bone graft are not displayed on an image screen of FIG. 10B.

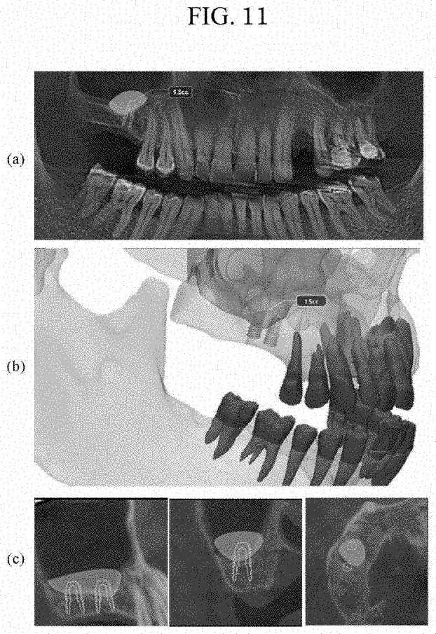

FIG. 11 is a view illustrating image screens for maxillary sinus lifting simulation according to one embodiment of the present disclosure.

Referring to FIG. 11, the software may display maxillary sinus lifting simulation information on all screens including: a panoramic image screen shown in FIG. 11A; a 3D image screen shown in FIG. 11B; and 2D cross-section image screens (axial, coronal, sagittal screens) shown in FIG. 11C. Here, the displayed maxillary sinus lifting simulation information includes information on an area where maxillary sinus involvement occurs based on the implant and information on a volume value thereof.

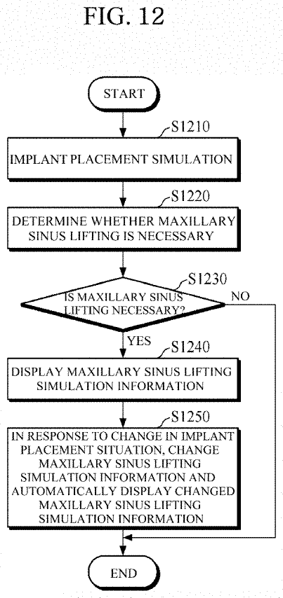

FIG. 12 is a view illustrating a flowchart of a maxillary sinus lifting simulation method according to one embodiment of the present disclosure.

Referring to FIG. 12, the software performs implant placement simulation in a dental image of a patient (S1210).

Then, during the implant placement simulation, the software determines whether maxillary sinus lifting is necessary due to maxillary sinus involvement by an implant (S1220). In the determining of whether the maxillary sinus lifting is necessary (S1220), the software may set a maxillary sinus simulation area based on the implant placed in the dental image. For example, the software sets the maxillary sinus simulation area in a semispherical shape having, as a radius, a length from a reference point of the implant as a start point to a point at a predetermined distance from an upper end of the implant. In a case in which two implants are consecutively placed, in response to two maxillary sinus simulation areas at least partially overlapping with each other, the software may connect end points of the two maxillary sinus simulation areas to each other to set a single maxillary sinus simulation area. Then, the software sets an implant involvement area within the set maxillary sinus simulation area. Here, the software may calculate a density value of the maxillary sinus simulation area and set an area having a density value less than a predetermined density value in the maxillary sinus simulation area as the implant involvement area. Then, the software determines whether the implant involves the implant involvement area and, in response to the implant involving the implant involvement area, determines that the maxillary sinus lifting is necessary.

In response to the maxillary sinus lifting being determined as necessary (yes in S1230), the software performs maxillary sinus lifting simulation and automatically displays maxillary sinus lifting simulation information in the dental image (S1240). The maxillary sinus lifting simulation information may include information on an area where maxillary sinus involvement occurs based on the implant and information on an amount of bone graft. The information on the amount of bone graft may be expressed as a volume value of the implant involvement area.

Then, in response to a change in an implant placement situation, the software may change the maxillary sinus lifting simulation information according to the changed implant placement situation and automatically display the changed maxillary sinus lifting simulation information in the dental image (S1250). For example, in response to a change in implant information, the software changes the information on the area where the maxillary sinus involvement occurs based on the implant and the information on the amount of bone graft to correspond to the changed implant information and automatically displays the changed information on the area where the maxillary sinus involvement occurs based on the implant and information on the amount of bone graft in the dental image. In response to movement of the implant, the software may move the maxillary sinus simulation area together, and, in response to a change in a length of the implant, the software may change the radius of the maxillary sinus simulation area. The software may also change the implant involvement area and the information on the amount of bone graft according to the change in the maxillary sinus simulation area.

The present disclosure has been described above on the basis of embodiments thereof. Those of ordinary skill in the art to which the present disclosure pertains should understand that the present disclosure can be implemented in modified forms within the scope not departing from essential characteristics of the present disclosure. Therefore, the embodiments disclosed herein should be considered as illustrative, instead of limiting. The scope of the present disclosure is shown in the claims below instead of the above description, and all differences within the scope equivalent to the claims should be interpreted as falling within the scope of the present disclosure.

1. A maxillary sinus lifting simulation method performed by a maxillary sinus lifting simulation apparatus, the maxillary sinus lifting simulation method comprising:
an operation of performing implant placement simulation in a dental image of a patient;
an operation of, during the implant placement simulation, determining whether maxillary sinus lifting is necessary due to maxillary sinus involvement by an implant;
an operation of automatically performing maxillary sinus lifting simulation only in a case in which the maxillary sinus lifting is necessary and automatically displaying maxillary sinus lifting simulation information, which includes information on an area where maxillary sinus involvement occurs based on the implant and information on an amount of bone graft, in the dental image; and
an operation of, in response to a change in an implant placement situation, changing the maxillary sinus lifting simulation information according to the changed implant placement situation and automatically displaying the changed maxillary sinus lifting simulation information in the dental image.

2. The maxillary sinus lifting simulation method of claim 1, wherein the operation of the determining of whether the maxillary sinus lifting is necessary includes:
an operation of setting a maxillary sinus simulation area based on the implant placed in the dental image;
an operation of setting an implant involvement area within the set maxillary sinus simulation area;
an operation of determining whether the implant involves the implant involvement area in an implant placement process; and
an operation of, in response to the implant involving the implant involvement area, determining that the maxillary sinus lifting is necessary.

3. The maxillary sinus lifting simulation method of claim 2, wherein the operation of the setting of the maxillary sinus simulation area includes an operation of setting the maxillary sinus simulation area in a semispherical shape having, as a radius, a length from a reference point of the implant as a start point to a point at a predetermined distance from an upper end of the implant.

4. The maxillary sinus lifting simulation method of claim 2, wherein:
in the operation of the setting of the maxillary sinus simulation area, in a case in which two implants are consecutively placed, in response to two maxillary sinus simulation areas at least partially overlapping with each other, end points of the two maxillary sinus simulation areas are connected to each other to set a single maxillary sinus simulation area; and
in the setting of the implant involvement area, a single implant involvement area that the two implant involve is set within the set single maxillary sinus simulation area.

5. The maxillary sinus lifting simulation method of claim 2, wherein the operation of the setting of the implant involvement area includes:
an operation of calculating a density value of the set maxillary sinus simulation area; and
an operation of setting an area having a density value less than a predetermined density value in the maxillary sinus simulation area as the implant involvement area.

6. The maxillary sinus lifting simulation method of claim 1, wherein the operation of the automatically displaying of the maxillary sinus lifting simulation information in the dental image includes:
an operation of displaying the implant involvement area in the dental image; and
an operation of calculating and displaying a volume of the implant involvement area.

7. The maxillary sinus lifting simulation method of claim 1, wherein, in the operation of the changing of the maxillary sinus lifting simulation information and the automatically displaying of the changed maxillary sinus lifting simulation information in the dental image, in response to a change in implant information, the information on the area where the maxillary sinus involvement occurs based on the implant and the information on the amount of bone graft are changed to correspond to the changed implant information and are automatically displayed in the dental image.

8. The maxillary sinus lifting simulation method of claim 1, wherein, in the operation of the changing of the maxillary sinus lifting simulation information and the automatically displaying of the changed maxillary sinus lifting simulation information in the dental image, in response to movement of the implant, the maxillary sinus simulation area is moved together, in response to a change in a length of the implant, the radius of the maxillary sinus simulation area is changed, and then the information on the area where the maxillary sinus involvement occurs based on the implant and the information on the amount of bone graft are changed to correspond to the movement of the maxillary sinus simulation area or the change in the radius and are automatically displayed in the dental image.

9. The maxillary sinus lifting simulation method of claim 1, further comprising an operation of providing a maxillary sinus lifting simulation setting screen for setting at least one of an interface for setting whether to use maxillary sinus lifting simulation, an interface for setting a display color of an implant involvement area, an interface for setting a length from an upper end of the implant to a maxillary sinus simulation area, an interface for setting opacity of the implant involvement area, and an interface for setting a reference point of the implant.

10. The maxillary sinus lifting simulation method of claim 1, further comprising:
an operation of receiving a manipulation signal for user setting relating to area showing and area hiding; and
an operation of, in a case in which an area showing setting is selected according to the user setting, displaying the implant involvement area and information on a predicated amount of bone graft in the dental image and, in a case in which an area hiding setting is selected, not displaying the implant involvement area and the information on the predicated amount of bone graft in the dental image.

11. A maxillary sinus lifting simulation apparatus comprising:
a data acquisition device configured to acquire a dental image of a patient;
a controller configured to perform implant placement simulation in the dental image of the patient, during the implant placement simulation, determine whether maxillary sinus lifting is necessary due to maxillary sinus involvement by an implant, automatically perform maxillary sinus lifting simulation only in a case in which the maxillary sinus lifting is necessary, provide maxillary sinus lifting simulation information, which includes information on an area where maxillary sinus involvement occurs based on the implant and information on an amount of bone graft, and, in response to a change in an implant placement situation, change the maxillary sinus lifting simulation information according to the changed implant placement situation; and
an output device configured to automatically display the maxillary sinus lifting simulation information in the dental image.

12. The maxillary sinus lifting simulation apparatus of claim 11, wherein the controller sets a maxillary sinus simulation area based on the implant placed in the dental image, sets an implant involvement area within the set maxillary sinus simulation area, and then determines whether the implant involves the implant involvement area in an implant placement process, and, in response to the implant involving the implant involvement area, determines that the maxillary sinus lifting is necessary.

13. The maxillary sinus lifting simulation apparatus of claim 11, wherein:
the controller sets the maxillary sinus simulation area in a semispherical shape having, as a radius, a length from a reference point of the implant as a start point to a point at a predetermined distance from an upper end of the implant, and
in a case in which two implants are consecutively placed, in response to two maxillary sinus simulation areas at least partially overlapping with each other, the controller connects end points of the two maxillary sinus simulation areas to each other to set a single maxillary sinus simulation area and sets a single implant involvement area that the two implant involve within the set single maxillary sinus simulation area.

14. The maxillary sinus lifting simulation apparatus of claim 11, wherein the output device displays the implant involvement area and a volume thereof in the dental image and, in response to a change in implant information, changes the implant involvement area and the volume thereof to correspond to the changed implant information and displays the changed implant involvement area and volume thereof in the dental image.

15. The maxillary sinus lifting simulation apparatus of claim 11, wherein the output device provides a maxillary sinus lifting simulation setting screen for setting at least one of an interface for setting whether to use maxillary sinus lifting simulation, an interface for setting a display color of the implant involvement area, an interface for setting a length from the upper end of the implant to the maxillary sinus simulation area, an interface for setting opacity of the implant involvement area, and an interface for setting the reference point of the implant.
